(19) United States
(12) Patent Application Publication
(10) Pub. No.: US 2025/0114407 A1
(43) Pub. Date: Apr. 10, 2025

(54) Topical Sports Cream Composition

(71) Applicant: Thomas Rhee, Tampa, FL (US)

(72) Inventor: Thomas Rhee, Tampa, FL (US)

(21) Appl. No.: 18/781,880

(22) Filed: Jul. 23, 2024

Related U.S. Application Data
(60) Provisional application No. 63/543,474, filed on Oct. 10, 2023.

(51) Int. Cl. A61K 35/51; A61K 31/05; A61K 31/10; A61K 31/135; A61K 35/50

(52) U.S. Cl. CPC A61K 35/51; A61K 31/05; A61K 31/10; A61K 31/135; A61K 35/50

(57) ABSTRACT

The present invention relates to topical sports cream composition including dimethyl sulfoxide (DMSO), capsaicin, amniotic membrane, umbilical cord, and menthol, and to methods of manufacture and methods of promoting tissue regeneration, increase recovery of damage tissue, activating tissues, increasing circulation, producing thermogenic effects and reaching full potential of tissues, and minimizing the risk of injuries with such composition.

### CROSS-REFERENCE TO RELATED APPLICATIONS

This application is a continuation of U.S. patent application Ser. No. 63/543,474 filed Oct. 10, 2023, the disclosures of which are hereby incorporated by reference in its entirety.

### FIELD OF THE INVENTION

The present invention is topical sports cream composition; and more specifically it enhances athletic performance and minimizes the risk of injuries by activating tissues, increasing circulation, producing thermogenic effects and reaching full potential of tissues and/or regenerate tissues with such composition.

### BACKGROUND

Over the past several years, people have been actively involved in the administration of stem cell injections to address sports-related injuries. However, this traditional approach to treatment presents certain challenges that hinder its widespread accessibility and effectiveness. Primarily, these issues encompass the necessity for medical facility-based injections, the inherent risk of infections associated with invasive procedures, and the relatively high cost per injection.

To overcome these challenges and provide an innovative solution to individuals seeking effective sports injury treatment, I embarked on the development of a groundbreaking topical cream with unique transdermal capabilities. The objective of this research and development effort is to create a product that can effectively deliver stem cells when applied to the skin's surface, thus rendering it possible to administer the therapy in the comfort of one's own home. This not only dramatically reduces the overall cost of treatment but also eliminates the potential risk of infection associated with invasive medical procedures, making it a safer and more accessible option for individuals seeking relief from sports-related injuries.

In essence, this topical cream composition bring advancement in the field of sports injury treatment or post exercise fatigueless and not limited to this, offering a novel and cost-effective alternative to traditional stem cell injections. By harnessing the power of transdermal delivery, this invention has the potential to revolutionize the way enhance athletic performance as well as sports injuries are treated, empowering individuals to take control of their recovery journey with convenience, affordability, and minimized health risks.

### DETAILED DESCRIPTION

It is to be understood that the aspects of the present invention as described below are not limited to specific compositions, methods or preparing such compositions, or uses thereof, as such may, of course, vary. It is also to be understood that the terminology used herein is for the purpose of describing particular aspects only and is not intended to be limiting.

It must also be noted that as used herein and in the appended claims, the singular forms “a”, “an”, and “the” include plural reference unless the context clearly dictates otherwise. Thus, for example, reference to an “organ” is a reference to one or more organs and equivalents thereof known to those skilled in the art, and so forth.

As used herein, the term “about” means plus or minus 10% of the numerical value of the number with which it is being used. Therefore, about 30% means in the range of 25%-35%.

As used herein, the term “athletic” means specifically used to denote participants in the sport of athletics, encompassing disciplines such as track and field, marathon running, swimming, football, basketball, cricket, and not limited to this. It serves as a collective identifier for individuals engaged in various athletic pursuits, encompassing a wide spectrum of physical culture across different sports.

As used herein, the term “regenerate tissue” means regenerative tissue growth in humans refers to the regrowth of lost tissues or organs in response to injury, distinct from wound healing or partial regeneration. While wound healing primarily involves closing the injury site with varying degrees of scar tissue, regenerative growth aims to fully restore the original tissue structure and function. Certain tissues, such as skin, the vas deferens, and large organs like the liver, exhibit a remarkable ability to regenerate efficiently.

As used herein, the term “thermogenic effects” means thermogenic refers to the tendency to produce heat. The term is commonly applied to drugs that increase heat through metabolic stimulation or to microorganisms that generate heat within organic waste. Nearly all enzymatic reactions in the human body are thermogenic, contributing to the basal metabolic rate.

In bodybuilding, athletes aiming to reduce body fat use thermogenic substances to increase their basal metabolic rate, thereby enhancing overall energy expenditure.

As used herein, the term “blood circulation” or “circulatory system” means a network of organs that includes the heart, blood vessels, and blood, all of which work together to circulate blood throughout the human body. This system encompasses the cardiovascular system, comprising the heart and blood vessels. The circulatory system is divided into two circuits: systemic circulation and pulmonary circulation.

Blood is a fluid made up of plasma, red blood cells, white blood cells, and platelets. It circulates throughout the body, delivering oxygen and nutrients to tissues and removing waste materials. Key nutrients transported by the blood include proteins and minerals. Additionally, the blood carries essential components such as hemoglobin, hormones, and gases like oxygen and carbon dioxide.

As used herein, the term “Centella asiatica”, also known as Indian pennywort, Asiatic pennywort, spadeleaf, coinwort, or gotu kola, is a perennial herbaceous plant belonging to the Apiaceae family. It is eaten as a culinary vegetable and utilized in traditional medicine. Centella comprises pentacyclic triterpenoids and trisaccharide derivatives, such as asiaticoside, brahmoside, asiatic acid, and brahmic acid (also known as madecassic acid). Additionally, it contains centellose, centelloside, and madecassoside.

The invention relates to compositions, typically topical sport cream is a pharmaceutical formulation to enhance athletic performance and minimize the risk of injuries. What sets this cream apart is its incorporation of advanced stem cell technology, making it a unique solution for athletes and active individuals.

The compositions of the invention include appreciable quantities of dimethyl sulfoxide (DMSO), menthol, Capsaicin, umbilical cord, amniotic membrane-derived bioactive biomolecules which improve the performance of the athlete and minimize the risk of injuries if it takes place during the game or exercise.

### Compounds of the Invention

Dimethyl Sulfoxide (DMSO) is primarily utilized as a topical analgesic, a carrier for pharmaceuticals in topical formulations, an anti-inflammatory agent, and an antioxidant. Its capacity to enhance the absorption rate of certain compounds through biological tissues, notably the skin, makes it a valuable component in various transdermal drug delivery systems.

The formulations of the invention include 1-50%, 10-40% by weight dimethyl sulfoxide.

Menthol, an organic compound, is either artificially produced or derived from the essential oils of various mint species such as corn mint or peppermint. Menthol induces the familiar cooling sensation on the skin by activating the cold-sensitive TRPM8 receptors through its chemical properties.

The formulations of the invention include 1-30%, 10-15% by weight menthol.

Capsaicin, a potent compound found in chili peppers of the Capsicum genus, acts as a chemical irritant and neurotoxin for mammals, including humans, causing a burning sensation upon contact with tissues. It serves as an analgesic in topical ointments and dermal patches, effectively alleviating pain.

The formulations of the invention include 0-10%, 1-5% by weight capsaicin.

The amniotic membrane stands out as a compelling grafting option for wounds due to its distinctive properties. These include anti-inflammatory effects, bacteriostatic properties, wound protection, reduced scarring, pain alleviation, and the ability to initiate epithelialization.

The formulations of the invention include about 10-50 ml, 20-40 ml by weight amniotic membrane.

The umbilical cord in medicine represents one of the most promising frontiers for tissue repair. Umbilical cord, along with stem cells derived from cord blood, holds immense potential as the largest available sources of stem cells globally. Within UC lies a diverse mix of stem and progenitor cells at various stages of lineage commitment. This unique composition positions umbilical cord as a compelling candidate for cell-based therapies and tissue engineering applications. The remarkable capacity of these cells for extensive self-renewal and their ability to differentiate into multiple lineages further underscores their significance in medical research and treatment.

The formulations of the invention include about 0.5-10 mg, 1-5 mg by weight umbilical cord.

The cornerstone of the cream composition lies in its utilization of stem cell technology, specifically sourced from Wharton's Jelly with MSC (Mesenchymal Stem Cells) and Amniotic Membrane Stem Cell-derived composition of exosomes, growth factors, Telomerase, other extracellular matrix and acellular derived biomolecules. These stem cells possess exceptional regenerative properties, initiating tissue repair and recovery when applied topically. This activation significantly enhances tissue flexibility, responsiveness, and overall performance during physical activity.

In addition to tissue activation, the topical sport cream continues to deliver thermogenic effects. The localized warmth generated by the cream serves to warm up targeted tissues, thereby reducing the risk of injuries and facilitating improved blood circulation.

Stem cell technology further contributes to heightened circulation by promoting angiogenesis—the growth of new blood vessels. Improved blood flow delivers increased oxygen and nutrients to the tissues, resulting in expedited tissue repair, reduced muscle fatigue, and heightened overall performance.

With the infusion of stem cell technology, the topical sport cream helps tissues attain their full potential. Stem cells, working synergistically with other active ingredients (described below), optimize tissue function, ensuring that muscles, tendons, ligaments, and not limited to this, are primed for movement, performance, and recovery. This topical cream uses the dimethyl sulfoxide (DMSO) as the transporter through the protected three layers of the skin to penetrants to the above-mentioned body parts or organs i.e., muscle, tendon, ligaments, joints, cartilages, etc.

The topical sport cream composition helps athletic performance. By harnessing the remarkable regenerative abilities of stem cells, this cream activates tissues, generates thermogenic effects, bolsters circulation, and maximizes tissue performance. It serves as a valuable tool for tissue repair, injury prevention, and post-exercise recovery.

The topical sport cream is a specialized formulation tailored for use before and after exercise or physical activity. This cream boasts advanced stem cell technology as its core component, offering a range of advantages to expedite recovery and promote tissue regeneration.

Incorporating stem cell technology into the topical sport cream enhances its ability to accelerate post-exercise recovery. Stem cells possess potent regenerative properties, aiding tissue repair and reducing recovery time. This enables athletes and active individuals to bounce back quickly and resume their activities with improved efficiency.

Stem cell technology plays a pivotal role in promoting tissue regeneration. Stem cells in the cream can differentiate into various cell types, including muscle and connective tissues, stimulating the body's natural repair mechanisms. This is vital for repairing micro-tears in muscle fibers and rejuvenating other damaged tissues, thus supporting overall tissue health.

In addition to regeneration, the topical sport cream facilitates the restoration of tissue function. Stem cells, along with other active ingredients, assist in the recovery of damaged tissues, helping them regain strength and elasticity. This restoration process ensures that tissues recover fully and are prepared for subsequent physical activities.

Following intense exercise, tissues may become fatigued and depleted of energy. The stem cell technology in the topical sport cream provides a rejuvenating effect by promoting cellular energy production. This helps tissues recover and re-energize for future workouts or activities.

Post-exercise tension and discomfort are common, and the topical sport cream addresses this with its stem cell technology and calming ingredients. This soothing effect contributes to an overall sense of well-being and readiness for the next physical challenge.

The topical sport cream, enriched with stem cell technology, is designed specifically for post-exercise recovery and tissue regeneration. Its unique combination of ingredients, including stem cells, works in synergy to expedite recovery, regenerate and restore damaged tissues, re-energize muscles, and provide calming effects.

By incorporating the topical sport cream into their post-activity routine, athletes and active individuals can optimize their recovery, minimize the risk of injuries, and maintain peak performance.

The ingredients, not limited to this, of the topical sport cream: Aloe Vera Extract, Arachidyl Alcohol, Arachidyl Glucoside, Behenyl Alcohol, Cetearyl Alcohol, Cetearyl Glucoside, Chamomile Extract, Citric Acid, Coconut oil, Cocos Nucifera oil, Double Helix Water, Ethylhexyglycerin, Glyceryl stearate, Grape Seed Oil, Honeysuckle Flower Extract, Lactobacillus Ferment, Linoleic Acid, Lysolecithin, Neopentyl Glycol Crosspolymer, Palmitoyl Oligopeptide, Peptides, Perflurodecalin, Perilla Ocymoides Seed Oil, Pullulan, Sclerotium & Xanthan Gum, Shea Butter, Sodium Hyaluronate, Tocopherol, Vanilla Planifolia Fruit Extract, White Tea Leaf Extract, dimethyl sulfoxide (DMSO), Capsaicin, Menthol, Centella asiatica, Wharton's jelly or umbilical cord, and Amniotic Membrane Stem Cell-derived composition.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims.

What is claimed is:

1. A method for the topical sport cream composition for human use, said cream composition comprising:
dimethyl Sulfoxide in an amount from about 1% to about 50% by weight of the composition;
menthol in an amount from about 1% to about 30% by weight of the composition;
capsaicin in an amount from about 0% to about 10% by weight of the composition;
umbilical cord in an amount from about 0.5 mg to about 10 mg by weight of the composition; and
amniotic membrane derived bioactive biomolecules in an amount from about 10 ml to about 50 ml by weight of the composition;
wherein:
said formulation is a topical sport cream to help with speeding up recovery or increase recovery of damage tissue, promoting tissue regeneration and minimizing the risk of injuries.

2. The method according to claim 1 wherein dimethyl Sulfoxide in an amount from about 10% to about 40% by weight of the composition;
menthol in an amount from about 10% to about 15% by weight of the composition;
capsaicin in an amount from about 1% to about 5% by weight of the composition;
umbilical cord in an amount from about 1 mg to about 5 mg by weight of the composition; and
amniotic membrane derived bioactive biomolecules in an amount from about 20 ml to about 40 ml by weight of the composition.
